(19) United States
(12) Patent Application Publication
(10) Pub. No.: US 2009/0068122 A1
(43) Pub. Date: Mar. 12, 2009

(54) Dentifrice Compositions for Treating Xerostomia

(76) Inventors: Shira Pilch, Highland Park, NJ (US); James Gerard Masters, Ringoes, NJ (US)

Correspondence Address: COLGATE-PALMOLIVE COMPANY, 909 RIVER ROAD, PISCATAWAY, NJ, 08855, US

(21) Appl. No.: 11/850,955

(22) Filed: Sep. 6, 2007

(51) Int. Cl. A61K 8/21; A61K 8/22; A61K 8/34; A61Q 11/00

(52) U.S. Cl. 424/52; 424/49; 424/53; 424/55

(57) ABSTRACT

This invention encompasses an oral composition for treating or preventing dry mouth with following benefits: antibacterial/anti-fungal efficacy, low irritancy, and moisture retention. The compositions can be a toothpaste, a mouth rinse or a spray and include one or more non-ionic surfactants, as foaming agents, one or more broad spectrum anti-microbial ingredient, such as chlorhexidine and cetylpyridinium chloride, and one or more oral surface adhesive polysaccharides that can encage cationic actives to help the deposition and retention of the agents onto oral surfaces and thus provide antimicrobial efficacy.

### BACKGROUND OF THE INVENTION

Dry mouth, clinically called xerostomia, is defined as a subjective feeling of dryness of the mouth. It is caused primarily by reduction of salivary secretion, but the underlying mechanism for such reduction varies from patient to patient. Depending on its severity, dry mouth can cause discomfort and lead to pathological conditions, such as caries and fungal infection, specifically oral candidiasis.

Thus, there exists a need for a dentifrice composition having both oral hygiene and dry mouth prophylactic and therapeutic properties, as well as having the ability to be used as a treatment for such maladies.

Accordingly, the invention describes an oral composition for dry mouth relief with the following benefits anti-bacterial/anti-fungal property; low irritancy; and moisture retention.

### SUMMARY OF THE INVENTION

The invention encompasses dentifrice compositions for treating or preventing dry mouth and at least one associated pathology that including one or more surfactants; one or more anti-microbial agents, one or more adhesives. The dentifrice compositions have oral hygiene treatment capabilities, as well as a prophylactic effect with regard to dry mouth, periodontal disease, and caries.

Accordingly in one embodiment, the invention encompasses a method of treating or preventing dry mouth and at least one associated pathology including administering to subject in need thereof an effective amount of an oral care composition including one or more non-ionic surfactants; one or more anti-microbial agents, one or more adhesives.

### DETAILED DESCRIPTION OF THE INVENTION

The invention encompasses a combination of antimicrobial and moisture retention agents, which composition has properties that provide dry mouth relief. Surprisingly, it has been found that the dentifrice compositions described herein can sterically immobilize cationic antimicrobial actives by forming a cage-like structure to maintain the bioavailability of the actives and thereby provide efficient delivery to the oral mucosa. The improved mucosal interaction can provide consumer perceived attributes due to the surface adhesion properties and surface coating of the compositions of the invention. The additional physical entrapment of the antimicrobial agent at the surface can lead to more substantive performance.

The present invention encompasses dentifrice compositions for alleviating or preventing xerostomia (i.e., dry mouth) and associated disorders of the oral cavity.

As used herein, term “dentifrice” or “dentifrices” refer to products which remain in the mouth for a relatively short period of time, in which they are intimately contacted with substantially all surfaces of the teeth, and are then removed. Non-limiting examples of such products include toothpastes, prophylactic pastes, tooth polishes, gels, professional gels and other related professional products, as well as mouth washes, sprays, mouth rinses, dental flosses, chewing gums, lozenges, tablets, edible food products, and the like.

In one embodiment, the compositions of the dentifrice of the present invention are toothpastes, mouth rinse, mouth sprays, and dental solutions. In another embodiment, the composition is toothpaste or a mouth rinse, and in another embodiment the dentifrice composition is toothpaste.

It has now been found that dry mouth and associated disorders of the oral cavity including cavity and infection, can be treated or prevented by administering an oral care dentifrice composition containing one or more non-ionic surfactants; one or more anti-microbial agents, and one or more adhesive polysaccharides.

Accordingly, the oral care dentifrice compositions of the invention are useful in dentifrice formulations and compositions in treating dry mouth and in the removal of plaque and calculus. While not intending to be held by any particular theory, it appears that the oral care dentifrice compositions can radically decrease, and even stop, dry mouth as well as plaque and calculus (tartar) buildup. It also appears that the oral care dentifrice compositions can stop the demineralization effect of caries and eliminate oral disease causing bacteria without detrimentally affecting the normal micro-flora in the mouth.

The dentifrice compositions of the invention include one or more surfactants or a mixture of compatible surfactants. Suitable surfactants are those which are reasonably stable throughout a wide pH range, for example, anionic, cationic, nonionic or zwitterionic surfactants. In an illustrative embodiment, the surfactant is a non-ionic surfactant useful as a foaming agent.

Suitable surfactants are described more fully, for example, in U.S. Pat. No. 3,959,458, to Agricola et al.; U.S. Pat. No. 3,937,807, to Haefele; and U.S. Pat. No. 4,051,234, to Gieske et al., which are incorporated herein by reference.

In certain embodiments, anionic surfactants useful herein include the water-soluble salts of alkyl sulfates having from 10 to 18 carbon atoms in the alkyl radical and the water-soluble salts of sulfonated monoglycerides of fatty acids having from 10 to 18 carbon atoms. Sodium lauryl sulfate, sodium lauroyl sarcosinate and sodium coconut monoglyceride sulfonates are examples of anionic surfactants of this type. Mixtures of anionic surfactants may also be utilized.

In certain embodiments, the invention encompasses the use of non-ionic or zwitterionic surfactants, such as betaine in pluronic in the place of SLS. These surfactants are also reportedly associated with lower irritancy to oral mucosa comparing to SLS which may benefit dry mouth sufferers.

In another embodiment, cationic surfactants useful in the present invention may be derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof.

Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants that can be used in the compositions of the invention may be compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

In certain embodiments, zwitterionic synthetic surfactants useful in the present invention may be derivatives of aliphatic quaternary ammonium, phosphonium, and sulfonium compounds, in which the aliphatic radicals can be straight chain or branched, and wherein one of the aliphatic substituents contains 8 to 18 carbon atoms and one contains an anionic water-solubilizing group, e.g., carboxy, sulfonate, sulfate, phosphate or phosphonate. Illustrative examples of the surfactants suited for inclusion into the composition include, but are not limited to, sodium alkyl sulfate, sodium lauroyl sarcosinate, cocoamidopropyl betaine and polysorbate 20, and combinations thereof.

The surfactant or mixtures of compatible surfactants can be present in the compositions of the present invention in an amount of about 0.1% to about 5.0%, in another embodiment about 0.3% to about 3.0% and in another embodiment about 0.5% to about 2.0% by weight of the total composition.

Antimicrobial and antibacterial agents included in the present dentifrice compositions may include, but are not limited to, triclosan (5-chloro-2-(2,4-dichlorophenoxy)-phenol); chlorhexidine; chlorohexidine digluconate (CHX); alexidine, hexetidine (HEX); sanguinarine (SNG); benzalkonium chloride; salicylanilide; domiphen bromide; cetylpyridiniumchloride (CPC); tetradecylpyridinium chloride (TPC); N-tetra-decyl-4-ethylpyridinium chloride (TDEPC); octenidine; delmopinol; octapinol, and other piperidino derivatives; nicin preparations; zinc/stannous ion agents; antibiotics such as augmentin, amoxicillin, tetracycline, deoxycycline, minocycline, and metronidazole; peroxide, such as cylium peroxide, hydrogen peroxide, and magnesium monoperthalate an its analogs; and analogs and salts of the above listed antimicrobial.

In certain illustrative embodiments, the antifungal property can be provided by one of the broad spectrum antimicrobial agents, namely CHX and CPC, and agents specifically against fungal infections, such as Nystatin, Amphotericin B, and Natamycin. In certain embodiments, the compositions treat bacterial infections associated with dry mouth condition.

The antimicrobial or antibacterial agent may be present in the compositions of the present invention in an amount of about 0.1% to about 5.0%, in another embodiment in an amount of about 0.3% to about 3.0% and in another embodiment of about 0.5% to about 2.0% by weight of the total composition.

In order to provide a moisture retention property, other surface coating/moisture retaining polymers can also be incorporated in the composition. The polymers include but not limited to the following families of compounds: silicone oils (such as dimethicone, are known to provide surface coating and moisture retention properties in skin care), and polysaccharides (such as CMC, Xanthan, and Carbopol), hyaluronic acid, dermatan sulfate, chondrotin sulfate, heparin, heparan sulfate and keratin sulfate.

In order to assist the deliver of an antimicrobial agent, for example, CHX and CPC, to oral surfaces, both compounds are sterically immobilized within one or combination of glycoaminoglycans (GAGs) molecule matrix by encapsulation or pre-emulsification before formulated in the final composition. They can form complex with GAGs through charge-charge interaction. GAG-CHX or GAG-CPC complex can subsequently deposit and retain on oral surface via high surface adhesive property of GAG. The deposited complex will slowly release CPC or CHX to provide antimicrobial effect.

The polysaccharide may be present in the compositions of the present invention of about 0.1% to about 5.0%, in another embodiment of about 0.3% to about 3.0% and in another embodiment of about 0.5% to about 2.0% by weight of the total composition.

A further component of the dentifrice composition of this invention are hydrophilic liquid vehicles, including but not limited to glycerin, propylene glycol, polyethylene glycol, and hydrophobic liquid vehicles such as triglyceride, diglyceride, and organic oils including mineral oil, essential oils, and fatty vegetable oils. Essential oils, as used herein, are natural substances which are extracted via distillation from tiny molecular sacs of each botanical, and are part of the plant's immune system and yet a separate substance created from the plant. The distillation process extracts the volatile oil from the plant parts. Such essential oils are highly concentrated extracts that contain hormones, vitamins and antiseptics that work on many levels. These hydrophilic and hydrophobic liquid vehicles can be used either singly or in combination and can be added in a proportion of about 2 to about 50 wt. % (in the case of compositions including liquid vehicles), especially of about 10 to about 35 wt. % based on the whole composition.

Using one or more of these liquid vehicles, the composition of the present invention for the oral cavity may be formulated into a use form such as gel, liquid, or paste.

The dentifrice composition of the invention also contains flavor components, typically in the form of natural flavors or aroma oils and/or herbal extracts and oils. These flavor components can serve not only to give a palatable flavor to the dentifrice composition, but can act as natural antibacterial agents and preservatives at the same time. The oils suitable for use in the present invention include, but are not limited to, citric oil, lemon oil, lime oil, lemongrass oil, orange oil, sweet orange oil, grapefruit oil, pomegranate oil, apricot oil extract, tangerine extract, tangelo oil, peppermint oil, spearmint oil, sage oil, rosemary oil, cinnamon oil, winter green oil, clove oil, eucalyptus oil, ginger oil, sassafras oil, menthol, arvensis mint oil, synthetic mint flavors and oils, carvone, eugenol, methyleugenol, methyl salicylate, methyl eugenol, thymol, anethole, millefolium extract, chamomile, lavender oil, myrrh, eugenol, tea tree oil, sage oil, mallow, limonene, ocimene, n-decyl alcohol, citronellol, alpha-terpineol, linalol, ethyllinalol, thyme, almond oil, nutmeg, and vanillin. Either one of these flavors or a mixture of two or more of these flavors can be used in the dentifrice composition. The dentifrice compositions of the present invention can also optionally contain sweeteners such as saccharin sodium, acesulfame potassium, glycyrrhizin, perillartine, thaumatin, aspartylphenylalanyl methyl ester and xylitol. The content thereof may be in an amount of about 3% to about 20% by weight, or about 4% to about 15% by weight, based on the total composition.

The dentifrice compositions may include one or more abrasives, which may be used in the practice of the invention including, but are not limited to, silica abrasives such as precipitated silicas having a mean particle size of up to about 20 microns, such as Zeodent 115®, marketed by J. M. Huber. The abrasive material should be one, which is compatible with the composition of interest and does not excessively abrade dentin. Suitable abrasives include for example, silicas including gels and precipitates; insoluble polymetaphosphate, hydrated alumina, resinous abrasives such as polymerized resins (e.g., ureas, melamines, cross-linked epoxides, phenolics, and the like), and mixtures thereof.

Useful abrasives also include sodium metaphosphate, potassium metaphosphate, tricalcium phosphate, dihydrated dicalcium phosphate, aluminum silicate, calcined alumina, bentonite or other siliceous materials, or combinations thereof. The silica abrasives polishing materials useful herein, as well as the other abrasives, generally have an average particle size ranging between about 0.1 and 30 microns, from between 5 and 15 microns. The silica abrasives can be from precipitated silica or silica gels, such as the silica xerogels described in U.S. Pat. No. 3,538,230, to Pader et al. and U.S. Pat. No. 3,862,307, to Digiulio, both incorporated herein by reference. Particular silica xerogels are marketed under the trade name Syloid® by the W. R. Grace & Co., Davison Chemical Division. The precipitated silica materials include those marketed by the J. M. Huber Corp. under the trade name Zeodent®, including the silica carrying the designation Zeodent 115 and 119. These silica abrasives are described in U.S. Pat. No. 4,340,583, to Wason, incorporated herein by reference.

Additional silica abrasives suitable for use with the present invention include but are not limited to silica gels, precipitated silicas, silicates, and hydrated silica. Silica gels suitable for use with the present invention are hydrogels, hydrous gels, xerogels, and aerogels, such as those known in the art and described in U.S. Pat. No. 6,440,397, which is incorporated herein by reference. Precipitated silicas are those known in the art, such as the suitable dentifrice-type precipitated silicas described in U.S. Pat. No. 5,589,160, the contents of which are incorporated by reference. Suitable silicates are any of those naturally occurring or synthetic silicates suitable for use with dentifrice compositions. These silica abrasives can be used singly or in combination. In certain embodiments, silica abrasive for use with the present invention includes silica gels. The silica abrasives can be used together with the calcium salt or in lieu of the calcium salt component.

Water can optionally be incorporated into the present toothpaste compositions of the present invention. Water used in the preparation of commercially suitable toothpastes should be deionized and free of organic impurities. Water can generally includes about 0% to about 40% by weight of the compositions herein.

The dentifrice compositions of the present invention can also optionally include anti-caries agents. Anti-caries agents are water-soluble fluoride ion sources. The number of such fluoride ions sources is great and well known to those of skill in the art, and includes those disclosed in U.S. Pat. No. 3,535,421, which is incorporated herein by reference. Fluoride ion source materials include sodium fluoride, potassium fluoride, sodium monofluorophosphate and mixtures thereof. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., incorporated herein by reference.

Representative fluoride ion sources include, but are not limited to, stannous fluoride, sodium fluoride, potassium fluoride, sodium monofluorophosphate, sodium fluorosilicate, sodium monfluorophosphate (MFP), ammonium fluorosilicate, as well as tin fluorides, such as stannous fluoride and stannous chloride, and combinations thereof. Certain particular embodiments include stannous fluoride or sodium fluoride as well as mixtures thereof.

In certain embodiments, the oral care strip or tape oral composition of the invention may also contain a source of fluoride ions or fluorine-providing ingredient in amounts sufficient to supply about 25 ppm to 5,000 ppm of fluoride ions.

Fluoride ion sources may be added to the compositions of the invention at a level of about 0.01% to 3.0% in one embodiment or of about 0.03% to 1.0%, by weight of the composition in another embodiment. The dosage of the individual strip or tape (i.e., a single dose) is about 0.0001 to 0.003% by weight, 0.0005 to 0.003% by weight, and in another embodiment about 0.001 to 0.02% by weight.

Dentifrice compositions of the present invention can also optionally include one or more anticalculus (anti-tartar) agents. Anticalculus agents which may be useful in the dentifrice compositions of the present invention include antimicrobials such as chlorhexidine, niddamycin, and triclosan, metals and metal salts such as zinc citrate, Vitamin C, bisphosphonates, triclosanpyrophosphates, pyrophosphates, polyphosphates, polyacrylates and other polycarboxylates, polyepoxysuccinates, ethyenediaminetetraacetic acid (EDTA), nitrilotriacetic acid and related compounds, polyphosphonates, and polypyrophosphates such as sodium hexametaphosphate, as well as other anticalculus agents known to those of skill in the art, such as those described in K. J. Fairbrother et al., “Anticalculus agents,” Journal of Clinical Periodontology Vol. 27, pp. 285 301 (2000).

Nutrients and vitamins can also optionally be added to the dentifrice compositions of the present invention. Such agents can include folates, retinoids (Vitamin A), Vitamin B (B₁-thyamin, B₂-riboflavin, B₃-niacine, B₅-pantothenic acid, B₆-pyridoxine, B₇-biotin, B₈/B₉/Bc-folic acid, B₁₂-cyanocobalamin), Vitamin C (ascorbic acid, sodium ascorbate), Vitamin E, Vitamin E analogs (dl-.alpha.-tocopherol acetate, tocopherol succinate, tocopherol nicotinate) and zinc.

A variety of miscellaneous additives can also be optionally formulated into the dentifrices of the present invention, such as tooth desensitizing agents (e.g., potassium and strontium salts), condensed anti-tartar agents such as sodium and potassium tetrapyrophosphate, whitening agents such as aluminum oxide and calcium peroxide, debriding agents such as sodium bicarbonate, pigments and dyes, such as Blue 15-C174160, Green 7-C174260, Reds 4-CI12085 and 40 CI16035, Yellows 115 CI47005:1 and 5 CI19140, and Carmine 5 CI16035), as well as additives such as mica and sparkles. As with the other optional dentifrice additives, use can be made of either one of these ingredients or a mixture of two or more of these ingredients in amounts appropriate for the dentifrice composition.

In addition to the above-described components, the dentifrice composition of the present invention can further contain a variety of optional ingredients and vehicles generally used for preparations for use in the oral cavity, such as dentifrices. These optional components include, but are not limited to, such components as abrasives, surfactants, thickening agents, buffers, humectants, preservatives, and antibiotic and anti-caries agents. All of these additives, described in further detail below, are generally usual and would be known to one of skill in the art.

Another optional component of the dentifrice compositions of the present invention is a humectant. The humectant serves to keep compositions such as toothpaste compositions from hardening upon exposure to air, and to give mouthwash and toothpaste compositions a moist feel to the mouth. Certain humectants can also impart desirable sweetness of flavor to toothpaste and mouthwash compositions. Suitable humectants for use in compositions of the present invention include edible polyhydric alcohols such as glycerin, sorbitol, xylitol, polyethylene glycol, and propylene glycol.

Buffering agents are another optional component of the dentifrice compositions of the present invention. The buffering agents serve to retain the pH of the compositions within the range. Suitable buffering agents for use in dentifrice compositions of the present invention include soluble phosphate salts.

Other optional components of the dentifrice compositions of the present invention are preservatives, such as those that prevent microbial growth in the dentifrice compositions. Suitable preservatives include, but are not limited to, methylparaben, propylparaben, benzoates, and alcohols such as ethanol.

Binders and thickeners can also optionally be used in the dentifrice compositions of the present invention, particularly in toothpaste compositions. In certain embodiments, binders and thickening agents include, but are not limited to, carrageenan (e.g., Viscarin, Irish moss, and the like); cellulose derivatives such as hydroxyethyl cellulose, sodium carboxymethyl cellulose, and sodium carboxymethyl hydroxypropyl cellulose, carboxyvinyl polymers; natural gums such as karaya gum, gum Arabic, and tragacanth; polysaccharide gums such as xanthan gum; fumed silica; and colloidal magnesium aluminum silicate.

It has been unexpectedly found that the dentifrice composition of the present invention has the ability to reduce, treat or prevent dry mouth. The composition according to the present invention may be used by applying it into the oral cavity and brushing the teeth and/or gingivae. For such brushing, use of a toothbrush provided with plaque removing bristles and gingiva-massaging portions in combination, although a conventional toothbrush, namely, a toothbrush having plaque removing bristles or a toothbrush having massaging portions may also be used.

### EXAMPLES

### Example 1

Illustrative Example 1 provides a non-limiting example of a toothpaste formulation. The following ingredients were combined in the proportions described below:

| Ingredients | % Wt./Wt. |
|---|---|
| Water | 3.78 |
| Sodium fluoride | 0.26 |
| Sodium saccharin | 0.37 |
| Sorbitol | 67.1 |
| Glycerine | 7.46 |
| PEG 600 | 1.5 |
| Tetra sodium | 0.53 |
| pyrophosphate | |
| Xanthan | 0.23 |
| Carboxymethyl cellulose | 0.8 |
| Silica | 12.5 |
| Hyaluronic acid | 0.5 |
| Pluronic | 1 |
| Betaine surfactant | 0.37 |
| Flavor | 1.1 |
| Carboxymethyl cellulose | 0.5 |
| Dimethicone | 2 |

### Example 2

Illustrative Example 2 provides a non-limiting example of a mouth rinse formulation. The following ingredients were combined in the proportions given below:

| Ingredients | % Wt./Wt. |
|---|---|
| Water | 84.41 |
| Sodium saccharin | 0.01 |
| Glycerine | 15 |
| Hyaluronic acid | 0.05 |
| Carboxymethyl cellulose | 0.08 |
| Pluronic | 0.1 |
| Flavor | 0.1 |
| Cetyl pyridinium chloride | 0.05 |
| Dimethicone | 0.2 |

1. A method of treating and; or preventing dry mouth and at least one associated pathology comprising administering to a subject in need thereof an effective amount of a composition comprising at least one surfactant; at least one anti-microbial agent; and at least one polysaccharide.

2. The method of claim 1, wherein the composition is suitable for oral administration.

3. The method of claim 1, wherein composition is a toothpaste.

4. The method of claim 1, wherein composition is a mouthwash.

5. The method of claim 1, wherein composition is an oral spray.

6. The method of claim 1, wherein the at least one associated pathology is chosen from a bacterial infection, a flingal infection, and a protozoal infection.

7. The method of claim 1 wherein the composition has low irritancy to the oral cavity.

8. The method of claim 1, wherein the at least one surfactant is a non-ionic surfactant.

9. The method of claim 1, wherein the at least one surfactant is a cocoamidopropyl beraine.

10. The method of claim 1, wherein the at least one anti-microbial agent is chosen from triclosan (5-chloro-2-(2.4-dichlorophenoxy )-phenol); chlorhexidine; chlorhexidine digluconate; alexidine, hexetidine; sanguinarine; benzalkonium chloride; salicylanilide; domiphen bromide; cetylpyridiniumchloride; tetradecylpyridinium chloride; N-tetra-decyl-4-ethylpyridinium chloride; octenidine; delmopinol; octapinol, and other piperidino derivatives; nicin preparations; zinc/stannous ion agents; antibiotics such as augmentin, amoxicillin, tetracycline, deoxycycline, minocycline, and metronidazole; peroxide, such as cylium peroxide, hydrogen peroxide, and magnesium monoperthalate; and analogs and salts of the above-listed anti-microbials.

11. The method of claim 1, wherein the at least one adhesive is chosen from silicone oils, polysaccharides, CMC, xanthan, carbopol, hyaluronic acid, dermatan sulfate, chondrotin sulfate, heparin, heparan sulfate and keratin sulfate.

12. The method of claim 1, wherein the composition further comprises at least one surface coating/moisture retaining polymer.

13. The method of claim 1 wherein the composition further comprises at least one fluoride source.

14. The method of claim 12, wherein the at least one associated pathology is caries.

15. The method of claim 1 wherein the composition further comprises at least one abrasive.

16. The method of claim 1, wherein the at least one anti-microbial agent comprises about 0.1 to about 5 wt. % of the composition.

17. The method of claim 1, wherein the at least one surfactant comprises about 0.1 to about 3 wt. % of the composition.

18. The method of claim 1, wherein the at least one adhesive comprises about 0.1 to about 3 wt. % of the composition.

19. A method of treating andlor preventing dry mouth and at least one associated pathology comprising administering to a subject in ned thereof an effective amount of a composition comprising at least one surfactant; at least one anti-microbial agent; and at least one polysaccharide: wherein the at least one anti-microbial acent is chosen from benzalkonium chloride; salicylanilide; domiphen bromide; cetylpyridiniumchloride; tetradecylpyridinium chloride; N-tetra-decy-4-ethylpyridinium chloride; octenidine; delmopinol; octapinol, and piperidino derivatives.

20. The method of claim 19, wherein the at least one associated pathology is a bacterial infection.

21. The method of claim 19, wherein the at least one associated pathology is a fungal infection.

22. The method of claim 19, wherein the at least one anti-microbial agent is cetylpyridiniumchloride.

23. The method of claim 1 wherein the anti-microbial agent is bound to the polysaccharide.

24. The method of claim 1 wherein the polysaccharide adheres to an oral surface.

25. The method of claim 1 wherein the polysaccharide comprises glycoaminoglycans.

26. The method of claim 1 wherein the anti-microbial agent is encapsulated within the polysaccharide.

27. The method of claim 1 wherein the anti-microbial agent and polysaccharide have a charge-charge interaction.

28. The method of claim 1 wherein the polysaccharide binds to the anti-microbial agent, and adheres to an oral surface and releases the anti-microbial agent when administered to the subject.
